(19) United States
(12) Patent Application Publication
(10) Pub. No.: US 2005/0283182 A1
(43) Pub. Date: Dec. 22, 2005

(54) Systems and methods for intraluminal delivery of occlusive elements

(75) Inventors: Ryan K. Pierce, Mountain View, CA (US); Ronald French, Santa Clara, CA (US); Emily Vu, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Concentric Medical, Inc., Mountain View, CA (US)

(21) Appl. No.: 10/873,408

(22) Filed: Jun. 21, 2004

(51) Int. Cl. A61M 29/00

(52) U.S. Cl. 606/200

(57) ABSTRACT

The present invention advantageously provides improved systems and methods for intraluminal delivery of implantable occlusive elements, particularly hydratable polymeric filaments. In particular, the systems and methods of the present invention minimize or avoid clumping or clogging a delivery catheter. This is accomplished by utilization of a reinforcing member or sheath that provides an advancing axial force along or over a length of the occlusive element. When the reinforcing member or sheath is pulled in a proximal direction, the occlusive element is advanced to a target site, which may comprise an aneurysm, vasculature of a tumor, arterio-venous malformations, fistulas, or burst blood vessels.

### BACKGROUND OF THE INVENTION

1. Field of the Invention

The present application relates generally to medical systems and methods. More particularly, the present application relates to systems and methods for intraluminal delivery of implantable occlusive devices.

Numerous persons experience some form of hemorrhagic stroke or blood vessel rupture in the brain. Vaso-occlusive devices are surgical implements or implants that are placed within the vasculature of the human body, typically via a catheter, either to block the flow of blood through a vessel making up that portion of the vasculature by formation of an embolus or to form such an embolus within an aneurysm stemming from the vessel. Other vascular abnormalities treated using such devices include arterio-venous malformations, fistulas, and burst blood vessels. Significantly, abnormal vasculature generated in the process of tumor growth may be treated using these vaso-occlusive devices.

The use of such devices has grown radically outside the use of treatment of the vasculature. Virtually any anatomical fluid vessel or opening has been treated or closed using devices of this type.

There are a variety of materials and vaso-occlusive devices commercially and medically in use. Perhaps the most well known of these devices is the Guglielmi Detachable Coil (GDC) shown in U.S. Pat. Nos. 5,122,136 and 5,354,295, both to Guglielmi et al. These patents and many more that follow it, describe a helically wound coil that is introduced to a treatment site in the body by use of a pusher wire that resembles a standard guide wire. The junction between the pusher wire and the coil is an electrolytically erodible joint that, upon application of a small current, will harmlessly erode in the human body separating the pusher wire from the coil. In overall summary, the procedure utilizing the GDC is this: the coil portion of the device is delivered by a catheter to the treatment site, the electricity is applied, the joint separates, the coil remains in the body forming the desired embolus, and the pusher wire and catheter are retrieved from the body. Many other variations of metallic coils are found in the patent literature and on the commercial marketplace. However, such coils are not entirely successful in achieving complete occlusion. For example, coil stiffness or coil pitch may leave voids at the treatment site resulting in recanalization, requiring follow-up procedures.

Another type of occluding material are embolic agents that are introduced into the human body in a liquid form where they are transformed either by precipitation from solution (e.g., U.S. Pat. No. 5,925,683 to Park) or by chemical reaction.

Another, more recently developed vaso-occlusive material involves biocompatible polymeric agents that are hydratable or gels. They may be introduced into treatment sites in the body much in the same way that the coils are although they typically must be handled in a somewhat different fashion because of the nature of their makeup. The polymers typically are quite slippery and may be damaged if handled with lack of care and understanding. In particular, pushing or injecting vaso-occluding materials into treatment sites requires careful handling in order to avoid various problems. For example, the vaso-occluding filaments or particles under compression may clump and clog a distal end or tip of the delivery catheter. This distal obstruction results from the vaso-occluding materials buckling within the delivery catheter as such materials are unable to carry compressive axial force. Further, such vaso-occluding materials exhibit limited tensile strength which may also affect desired advancement, positioning, and retractability of such occlusion materials at a treatment site.

For these reasons, it would be desirable to provide improved systems and methods for intraluminal delivery of implantable occlusive devices, particularly hydratable polymeric filaments. The systems and methods should avoid clumping or clogging of the delivery apparatus. In particular, the methods and systems should provide for an advancing axial force along a length of the occlusive implant. It would be further desirable to provide systems and methods that enhance the mechanical integrity of the vaso-occluding filaments. At least some of these objectives will be met by the systems, methods, and kits of the present invention described hereinafter.

2. Description of the Background Art

Endovascular therapies for treating vessel ruptures and blood flow abnormalities include implanting vaso-occlusive agents, coils and other devices such as that described in U.S. Pat. No. 4,994,069, and injecting hydrogel vaso-occluding particles and filaments into the vessels to be treated, as described in U.S. patent application Publication Nos. 2002/0193812A1; 2002/0193813A1; 2003/004533A1; 2003/0004568A1; and 2002/0193812A1, as well as U.S. patent application Ser. Nos. 10/400,185 and 10/739,900, each of which are assigned to the assignee of the present application. U.S. Pat. Nos. 5,122,136; 5,354,295; and 5,925,683 have been described above. U.S. Pat. No. 6,299,590 describes methods and devices for inserting a ball-shaped implant made from pliable fibers. U.S. Pat. No. 6,312,421 describes the delivery of a biocompatible polymeric string to an aneurysm where the string is cut when the aneurysm is substantially filled.

The full disclosures of each of the above mentioned references are incorporated herein by reference in their entirety.

### BRIEF SUMMARY OF THE INVENTION

The present invention advantageously provides improved systems and methods for intraluminal delivery of implantable occlusive devices, particularly hydratable polymeric filaments. It is to be understood that the occluding filaments described herein are not limited to occluding blood vessels or aneurysms. Rather, the occluding materials described herein may be used to form an occlusion in any of the vessels, ducts, and cavities found in the body including but not limited to vessels found in the blood vasculature. In particular, the systems and methods of the present invention minimize or avoid clumping or clogging a delivery catheter or sheath.

In a first aspect of the present invention, a method for intraluminal delivery of an occlusive element is provided. A delivery sheath is positioned in a body lumen, such as a blood vessel. The delivery sheath may comprise a single lumen having at least an insertion lumen therethrough and containing at least one occlusive element and a reinforcing member incorporating the occlusive element along a length thereof. Incorporation may include coupling, affixing, adhering, or embedding the reinforcing member along a length of the occlusive element. Alternatively, the delivery sheath may comprise a dual lumen further including a return lumen. In either embodiment, the reinforcing member is pulled in a proximal direction, whether over an outside surface of the delivery sheath, through the return lumen, or via both embodiments. This in turn causes the occlusive element to be advanced to a target site, which may comprise an aneurysm, vasculature of a tumor, an arterio-venous malformation, fistula, or burst blood vessel.

The pulling action of the reinforcing member advantageously provides an advancing axial force along a length of the occlusive element to prevent the occlusive element from buckling and obstructing or clogging a distal end or tip of the delivery sheath. Pulling further comprises detaching the reinforcing member from the occlusive element at a distal end of the insertion lumen so as to advance the occlusive element distally of the delivery sheath for proper positioning at the target site. The method further comprises hydrating the filament, generally prior to advancing the occlusive element to the target site, so as to form a polymeric gel. Additionally, in certain circumstances such as inadvertent advancement into a non-target parent vessel, the occlusive element may be retracted through the insertion lumen via the reinforcing member.

In another aspect of the present invention, a system for intraluminal delivery of an occlusive element is provided comprising a delivery sheath, at least one occlusive element, and at least one reinforcing member. The delivery sheath may comprise a single lumen structure having an insertion lumen. In other instances, the delivery sheath may comprise a dual lumen structure having both an insertion lumen and a return lumen, wherein the insertion lumen and return lumen are separated or concentric with respect to each other. The at least one occlusive element is slidably received within the insertion lumen. The at least one reinforcing member is embedded along a length of the occlusive element. Depending on the single or double lumen structure of the delivery sheath, the reinforcing member may be slidably received within the return lumen or disposed over an outside surface of the delivery sheath.

As noted above, the reinforcing member provides an axial force along a length of the occlusive element as it is pulled proximally. As the reinforcing member is detached from the occlusive element at a distal end of the insertion lumen, the occlusive element is advanced distally of the delivery sheath. Detachment may be effected in several ways. For example, there may be an opening in a distal wall of the insertion lumen which allows for the reinforcing member to pass through yet is sufficiently small enough to prevent passage of the occluding member. In other instances, the reinforcing member may simply be inverted at a distal end of the insertion lumen or may pass through a slit running proximally from a distal end of the delivery sheath.

The reinforcing member may be formed from a variety of medical grade polymers including nylon, polyurethane, polyimide, polyester, polypropylene, polyethylene, silk, PTFE (polytetrafluoroethylene), ePTFE (expanded polytetrafluoroethylene), PET (polyethyleneterephthalate), CRISTAMID®, GRILAMID®, PEBAX®, and like threads or materials. The reinforcing member may be centrally embedded within the occlusive element or offset from a central axis of the occlusive element. Integration or inclusion of the reinforcing member may be achieved preferably during the occlusive element extrusion process or after filament extrusion. Generally, a length of the reinforcing member is about at least twice the length of the delivery sheath so that the reinforcing member may be evacuated and the occlusive element properly advanced. The reinforcing member will generally have a length of at least about 10 cm, and a diameter in a range from about 0.00025 inch to about 0.025 inch, preferably from about 0.0005 inch to about 0.005 inch.

The occlusive element, the composition of which is discussed in more detail in U.S. patent application Publication Nos. 2002/0193812A1 and 2002/0193813A1, assigned to the assignee of the present application, generally comprises a polymeric filament that is hydratable so as to form a polymeric gel. The occlusive element may form a final hydrated noodle shape having a length of at least about 0.5 cm, preferably in a range from about 2 cm to about 200 cm, and a diameter in a range from about 0.004 inch to about 0.125 inch, preferably from about 0.005 inch to about 0.025 inch. It is to be understood that the occlusive element may also take on a variety of other filament and particle shapes, sizes, and forms. It will further be appreciated that certain mechanisms or features may be built into the structure of the delivery sheath, catheter, occlusive element, or reinforcing member that will cooperate in some fashion to cause or to permit severing or detaching the occlusive element for its release at the target site.

In some embodiments, a core member may additionally be embedded along a length of the occlusive element, as discussed in more detail in U.S. patent application Publication No. 2003/0004568A1, assigned to the assignee of the present application. The core member may comprise metal materials, such as stainless steel, shape memory alloy, or superelastic metal, or polymer materials, such as nylon, polyurethane, polyimide, polyester, polypropylene, polyethylene, silk, PTFE (polytetrafluoroethylene), ePTFE (expanded polytetrafluoroethylene), PET (polyethyleneterephthalate), CRISTAMID®, GRILAMID®, PEBAX®, and like materials. The core member enhances the mechanical integrity, such as the tensile strength, of the occlusive element which in turn improves the desired advancement, positioning, and retractability of such occlusion materials at a treatment site. The core member will generally have a length of at least about 0.5 cm, preferably in a range from about 2 cm to about 500 cm.

The delivery sheath may be slidably received within a conventional catheter structure, and in some instances the delivery sheath and catheter may form an integrated unit. The delivery sheath and catheter may be formed from a variety of medical grade materials, such as polymer tubes. In some instances, the polymer tubes may be reinforced with a braided metal or polymer, and/or lined with a low-friction material such as PTFE (polytetrafluoroethylene) or polyethylene, and/or coated with low-friction, hydrophilic coatings, such as hyaluronan-based coatings (e.g., HYDAK®) or polyvinylpyrrolidone-based coatings. The delivery sheath will generally have a length in a range from about 5 cm to about 300 cm, preferably from about 100 cm to about 250 cm.

In a further aspect of the invention, another method for intraluminal delivery of an occlusive element is provided. A delivery catheter is positioned in a body lumen. The catheter will generally comprise at least an insertion lumen and a return lumen therethrough. The catheter will further contain at least one occlusive element and a reinforcing sheath encompassing at least a portion of the occlusive element along a length thereof. The reinforcing sheath will be pulled in a proximal direction through the return lumen. This advancing force in turn will advance the occlusive element to the target site.

In this embodiment, pulling the reinforcing sheath instead provides an advancing shear force to an outside surface of the occlusive element along a length thereof to prevent the occlusive element from buckling and obstructing or clogging a distal end or tip of the delivery catheter. This design further preserves the mechanical integrity of the occlusive element and may easily be incorporated into a delivery catheter structure. Pulling further comprises inverting the reinforcing sheath at a distal end of the insertion lumen so as to advance the occlusive element distally of the delivery catheter for proper positioning at the target site. Additionally, in certain circumstances such as inadvertent advancement into a non-target parent vessel, the occlusive element may be retracted through the insertion lumen via the reinforcing sheath.

In a still further aspect of the present invention, another system for intraluminal delivery of an occlusive element is provided comprising a delivery catheter, at least one occlusive element, and at least one reinforcing sheath. The delivery catheter generally comprises a dual lumen structure having both an insertion lumen and return lumen therethrough. The at least one occlusive element is slidably received within the insertion lumen. The at least one reinforcing sheath encompasses the occlusive element along a length thereof and is slidably received within both the insertion and return lumens.

The reinforcing sheath will have a longitudinal opening along a length thereof so as to allow for inversion of the reinforcing sheath as it is pulled proximally. The reinforcing sheath may comprise a variety of shapes. For example, the reinforcing sheath may comprise a circumferential arc in a range from about 180 degrees to about 360 degrees, preferably from about 270 degrees to about 340 degrees. The reinforcing sheath will have a length in a range from about 10 cm to about 600 cm, preferably from about 100 cm to about 500 cm. Generally, the length of the reinforcing sheath is at least twice the length of the delivery catheter so that the occlusive element is properly advanced as the reinforcing sheath is migrated. The reinforcing sheath may be formed from a variety of medical grade materials, including nylon, polyurethane, polyimide, polyester, polypropylene, polyethylene, silk, PTFE (polytetrafluoroethylene), ePTFE (expanded polytetrafluoroethylene), PET (polyethyleneterephthalate), CRISTAMID®, GRILAMID®, PEBAX®, and like materials.

The delivery catheter will generally have a length in a range from about 5 cm to about 300 cm, preferably from about 50 cm to about 250 cm, and a diameter in a range from about 0.025 inch to about 0.250 inch, preferably from about 0.030 inch to about 0.100 inch. It will be appreciated that the insertion lumen and return lumen preferably form concentric tubular members that may have varying lengths. In some instances, the insertion lumen and return lumen may merge as interference between insertion and return of the reinforcing sheath may be negligible. In such a case, walls of the insertion lumen and return lumen (i.e., inner and outer shaft) may be bridged to form an integrated unit at a distal end of the delivery catheter.

The present invention further includes kits comprising an occlusive element delivery system as described herein and instructions to use the system for hemostasis of a puncture site in a blood vessel. Instructions for use will generally recite the steps for performing one or more of the above described methods. The instructions will often be printed, optionally being at least in part disposed on packaging. The instructions may alternatively comprise a videotape, a CD-ROM or other machine readable code, a graphical representation, or the like showing any of the above described methods. The kit may further include additional components of the system, such as hydrating fluid source, or the like. The kit components will be packaged in a conventional medical device package that is usually sterile, such as a pouch, tray, box, or the like.

A further understanding of the nature and advantages of the present invention will become apparent by reference to the remaining portions of the specification and drawings.

### BRIEF DESCRIPTION OF THE DRAWINGS

The following drawings should be read with reference to the detailed description. Like numbers in different drawings refer to like elements. The drawings, which are not necessarily to scale, illustratively depict embodiments of the present invention and are not intended to limit the scope of the invention.

FIG. 1 illustrates a perspective view of a typical catheter assembly containing a delivery sheath and an occlusive element sticking out from one end.

FIG. 2 illustrates a partial cutaway of introduction of an occlusive implant into an aneurysm in the vasculature using a catheter.

FIGS. 3A through 3C illustrate side and cross sectional views of a single lumen delivery sheath constructed in accordance with the principles of the present invention.

FIGS. 4A through 4C illustrate side and cross sectional views of a dual lumen embodiment of the delivery sheath.

FIGS. 5A through 5C illustrate side and cross sectional views of a further dual lumen embodiment of the delivery sheath.

FIG. 6 illustrates a side view of a still further dual lumen embodiment of the delivery sheath.

FIG. 7 illustrates another embodiment of the occlusive element disposed within a concentric delivery sheath.

FIG. 8 illustrates a method for intraluminal delivery of an occlusive element into an aneurysm using the delivery sheath of FIG. 4.

FIG. 9 illustrates a side view of a delivery catheter constructed in accordance with the principles of the present invention.

FIGS. 10A through 10C illustrate cross sectional views of a distal end of the delivery catheter of FIG. 9.

FIG. 11 illustrates a side view of an alternative embodiment of the delivery catheter.

FIGS. 12A through 12C illustrate cross sectional views of a distal end of the delivery catheter of FIG. 11.

FIG. 13 illustrates a method for intraluminal delivery of an occlusive element into an aneurysm using the delivery catheter of FIG. 9.

### DETAILED DESCRIPTION OF THE INVENTION

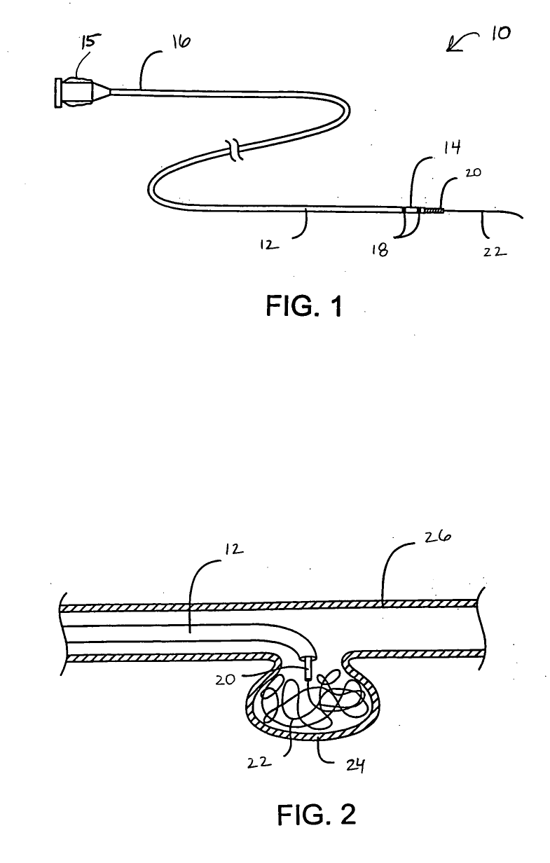

Typically, the occlusive device or element described herein will be delivered using a catheter assembly, e.g. (10) as shown in FIG. 1. Catheters are well known devices for delivering occlusive devices into the vasculature. They are thoroughly designed and many variations are available for reaching various regions in the vasculature whether the selected site for treatment be in a large vessel such as the descending aorta or in the fine and narrow vasculature of the brain. Shown in FIG. 1 is a catheter (12) that often is constructed in such a way that the distal end (14) of the catheter (12) is significantly less stiff than the proximal end (16). When the catheter (12) is small, e.g., because it is to be used in the neurovasculature, this is especially true. The proximal construction of the assembly generally includes a conventional hub (15) coupled to the proximal end (16). Also shown in FIG. 1 are radio-opaque markers (18) that allow the end of the catheter to be readily observed using fluoroscopy. The delivery sheath (20) is also shown as is the filamentary occlusion device (22). The delivery sheath (20) and the occlusive element (22) will be discussed in more detail below.

FIG. 2 shows the placement of a catheter (12) such as was shown in FIG. 1 as it is used in providing a pathway for the delivery sheath (20) and the occluding element (22). In FIG. 2, the occlusive element (22) is used to fill an aneurysm (24) that extends from a parent vessel (26). It will be appreciated that the above depictions are for illustrative purposes only and do not necessarily reflect the actual shape, size, or dimensions of the system (10). This applies to all depictions hereinafter.

This system may deliver one or more occlusive elements. Typically, the occlusive element will comprise filamentary shapes. Of particular interest are filaments comprising natural or synthetic polymeric hydratable gel. Synthetic polymers may be, for instance selected from the group consisting of polyacrylamide (PAAM), hydrophilic polyacrylonitrile (HYPAN), poly(N-isopropylacrylamine) (PNIPAM), poly(vinylmethylether), poly(ethylene oxide), poly(vinylalcohol), poly(ethyl(hydroxyethyl) cellulose), poly(2-ethyl oxazoline), polylactide (PLA), polyglycolide (PGA), poly(lactide-co-glycolide) PLGA, poly(e-caprolactone), polydiaoxanone, polyanhydride, trimethylene carbonate, poly((ÿ-hydroxybutyrate), poly(g-ethyl glutamate), poly(DTH-iminocarbonate), poly(bisphenol-A iminocarbonate), poly(orthoester) (POE), polycyanoacrylate (PCA), polyphosphazene, polyethylene oxide (PEO), polyethyleneglycol (PEG), polyacrylic acid (PAA), polyacrylonitrile (PAN), polyvinylacrylate (PVA), polyvinylpyrrolidone (PVP), polyglycolic lactic acid (PGLA), their block and random copolymers, and their blends. Natural polymers, for instance, may be materials selected from the group consisting of collagen, silk, fibrin, gelatin, hyaluron, cellulose, chitin, dextran, casein, albumin, ovalbumin, heparin sulfate, starch, agar, heparin, alginate, fibronectin, fibrin, keratin, pectin, elastin, and their block and random copolymers and their blends. In addition, the occlusive elements may contain or be coated with one or more bioactive agents in an amount effective to provide or to promote a selected biological activity and may contain one or more radio-opacifiers.

The bioactive agent typically is selected to provide or to promote a biological activity at the occlusive device's selected implantation site. For instance, the bioactive agent may be selected from the group consisting of compositions that occlude blood flow, adhere to the occlusive device at the site, rebuild damaged vascular wall, regress or inhibit capillary dilation, regress or inhibit venous malformation, and regress or inhibit tumor growth at or near the implantation site.

By way of further example, the bioactive agent may be selected from the group consisting of protein factors, growth factors, inhibiting factors, endothelization factors, extracellular matrix-forming factors, cell adhesion factors, tissue adhesion factors, immunological factors, healing factors, vascular endothelial growth factors, scarring factors, tumor suppression antigen-binding factors, anti-cancer factors, monoclonal antibodies, monoclonal antibodies against a growth factor, drugs, drug producing cells, cell regeneration factors, progenitor cells of the same type as vascular tissue, and progenitor cells that are histologically different from vascular tissue.

The term “an effective amount of” a given agent or agents is to be determined on an agent-by-agent basis, taking into account such standard, known parameters of bioactive agents such as potency, available concentration, and volume of space within the patient to be targeted for the desired effect. Efficacy and proper dosage are determined by routine assays specific for the bioactive agent selected using, for example, standard assays found in well known and frequently used laboratory assay and protocol manuals for identifying activity and quantifying potency of molecules and cells.

The occlusive elements may also comprise a radio-opacifier, e.g., a material that provides visibility of the device under X-ray or other imaging technology such as computer assisted tomography (CT scans), magnetic resonance imaging (MRI's), and fluoroscopy. For instance, a selected radio-opacifier may include a gadolinium based MRI contrast agent. These agents may include gadopentetate, gadopentetate dimeglumine (Gd-DTPA sold as “Magnevist”), gadoteridol (Gd HP-1303A sold as “ProHance”), gadodiamide (Gd-DTPA-BMA sold as “Omniscan”), gadoversetamide (Gd-DTPA-BMEA sold as “OptiMARK”), Gd-DOTA (sold as “Magnevist” or “Iotarem”), Gd-DTPA labeled albumin, and Gd-DTPA labeled dextran. Other suitable fluoroscopic radio-opacifiers include those that are variously soluble in the polymer precursors or the polymer itself, e.g., metrizamide (see, U.S. Pat. No. 3,701,771) or iopromide (see, U.S. Pat. No. 4,364,921—often sold in a dilute form under the tradename “Ultravist”) and solid, powdered materials such as barium sulfate, bismuth trioxide, bismuth carbonate, tungsten metal, and tantalum metal, and the like. Other iodine based and powdered metal-based radio-opacifiers are also well-known.

The bioactive agents and radio-opaque materials may be integrated into the typically extruded occlusive elements. Integration or inclusion of the bioactive agents and radio-opaque materials into the extruded product may be accomplished during extrusion or after extrusion. Such integration may be accomplished after extrusion such as by the acts consisting of coating, dipping, jacketing, spraying, weaving, braiding, spinning, ion implantation, vapor deposition, and plasma deposition. Integration of the bioactive agents and radio-opaque materials during extrusion may also be accomplished by placing the agent into a solvent used to dissolve the polymeric material making up the occluding filament. The bioactive agents and radio-opaque materials may (depending upon their composition) also be incorporated into the filament during subsequent hydration of the extruded filament.

It will be appreciated that the composition of the occlusive element may vary along its length and may well have certain features built into the structure that will cooperate in some fashion to cause or to permit severing or detaching it. Alternatively or in addition, certain mechanisms may be built into the structure of the delivery sheath or catheter that will cooperate in some fashion to cause or to permit severing or detaching the occlusive element for its release at the target site.

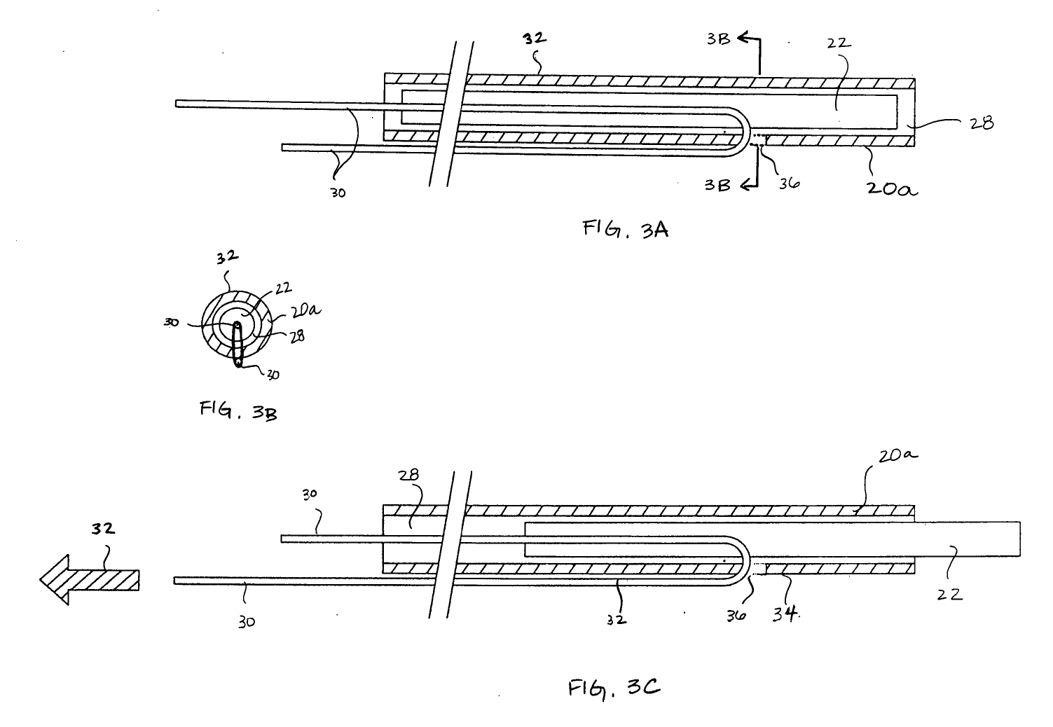

Referring now to FIGS. 3A through 3C, side and cross sectional views of a single lumen delivery sheath (20a) constructed in accordance with the principles of the present invention are illustrated. The delivery sheath (20a) comprises a single insertion lumen (28). The at least one occlusive element (22) is slidably received within the insertion lumen (28). At least one reinforcing member (30) is embedded along a length of the occlusive element. In particular, the reinforcing member (30) is shown as being slidably received within the insertion lumen (28) and disposed over an outside surface (32) of the delivery sheath (20a).

The reinforcing member (30) provides an axial force along a length of the occlusive element (22) as the reinforcing member (30) is pulled proximally, as denoted by arrow 32 in FIG. 3C. As the reinforcing member (30) is detached from the occlusive element (22) at a distal end (34) of the insertion lumen (28), the occlusive element (22) is advanced distally of the delivery sheath (20a). Detachment is effected by an opening (36) in a distal wall (34) of the insertion lumen (28) which allows for the reinforcing member (30) to pass through yet is sufficiently small to prevent passage of the occluding member (22).

The reinforcing member (30) may be formed from a variety of medical grade polymers including nylon, polyurethane, polyimide, polyester, polypropylene, polyethylene, silk, PTFE (polytetrafluoroethylene), ePTFE (expanded polytetrafluoroethylene), PET (polyethyleneterephthalate), CRISTAMID®, GRILAMID®, PEBAX®, and like threads or materials. As shown in the cross-section view of FIG. 3B, the reinforcing member (30) is centrally embedded within the occlusive element (22). Integration or inclusion of the reinforcing member (30) may be achieved preferably during the occlusive element (22) extrusion process. For example, such integration of the reinforcing member during extrusion may be accomplished by placing the reinforcing member into a solvent used to dissolve the polymeric material making up the occluding element.

Generally, a length of the reinforcing member (30) is at least twice the length of the delivery sheath (20) so that the reinforcing member (30) may be evacuated and the occlusive element (22) properly advanced. The reinforcing member (30) will generally have a length of at least about 10 cm. The reinforcing member (30) will generally have a diameter in a range from about 0.00025 inch to about 0.025 inch, preferably from about 0.0005 inch to about 0.005 inch.

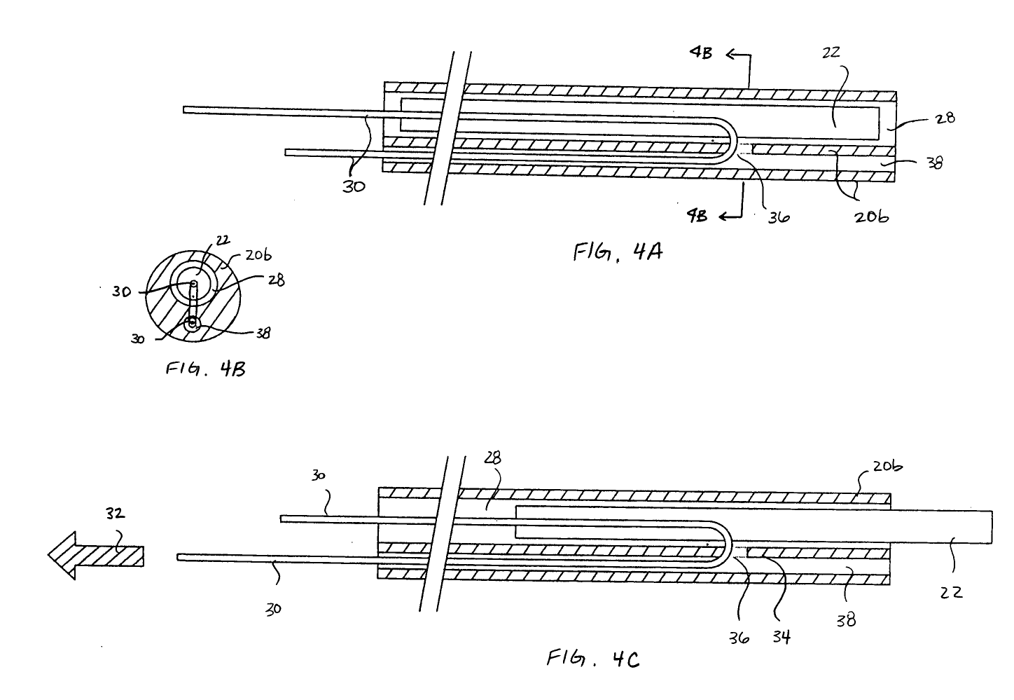

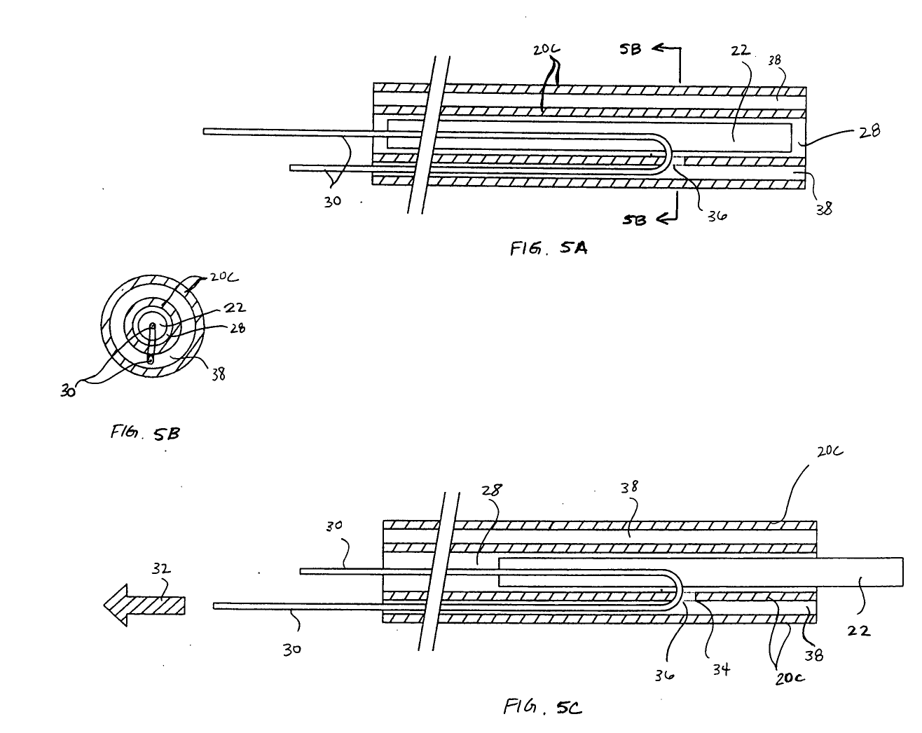

Referring now to FIGS. 4A through 4C, side and cross sectional views of yet another delivery sheath (20b) constructed in accordance with the principles of the present invention are illustrated. The delivery sheath (20b) comprises a double lumen structure having an insertion lumen (28) and a smaller return or second lumen (38). The lumens are separated with respect to each other as depicted in the cross sectional view of FIG. 4B. The at least one occlusive element (22) is slidably received within the insertion lumen (28). The reinforcing member (30) is embedded along a length of the occlusive element (22). In particular, the reinforcing member (30) is shown as being slidably received within both the insertion lumen (28) and return lumen (38) of the delivery sheath (20a) as it is pulled in a proximal direction, as denoted by arrow (32) in FIG. 4C. In this embodiment, the opening (36) in the distal wall (34) of the insertion lumen (28) extends to the return lumen (38) so as to allow the reinforcing member (30) to detach from the occlusive element (22).

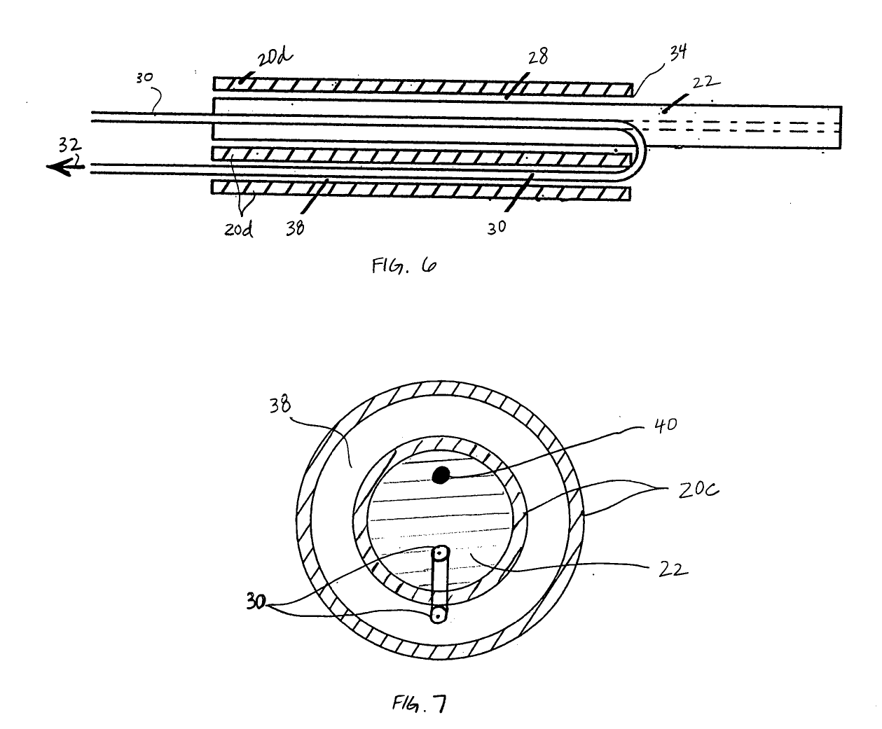

Referring now to FIGS. 5A through 5C, another double lumen delivery sheath (20c) is illustrated having both an insertion lumen (28) and a return lumen (38). In this embodiment, the insertion lumen (28) and return lumen (38) are concentric with respect to each other as shown in FIG. 5B. The reinforcing member (30) is pulled proximally through the opening (36) in the distal wall (34) of the insertion lumen (28) and through the annular space between the lumens. It will be appreciated that in these double lumen embodiments, the insertion lumen (28) and return lumen (38) will be stationary with respect to each other and that the lumens may comprise varying lengths. In some instances, the insertion lumen and return lumen may merge at a proximal end and the lumen walls bridged together at a distal end of the delivery sheath or catheter (not shown).

Referring now to FIG. 6, another double lumen delivery sheath (20d) similar to FIG. 4 is illustrated. The delivery sheath (20d) of FIG. 6 however has no opening, but rather detachment is effected by simply inverting the reinforcing member (30) at a distal end (34) of the insertion lumen (28).

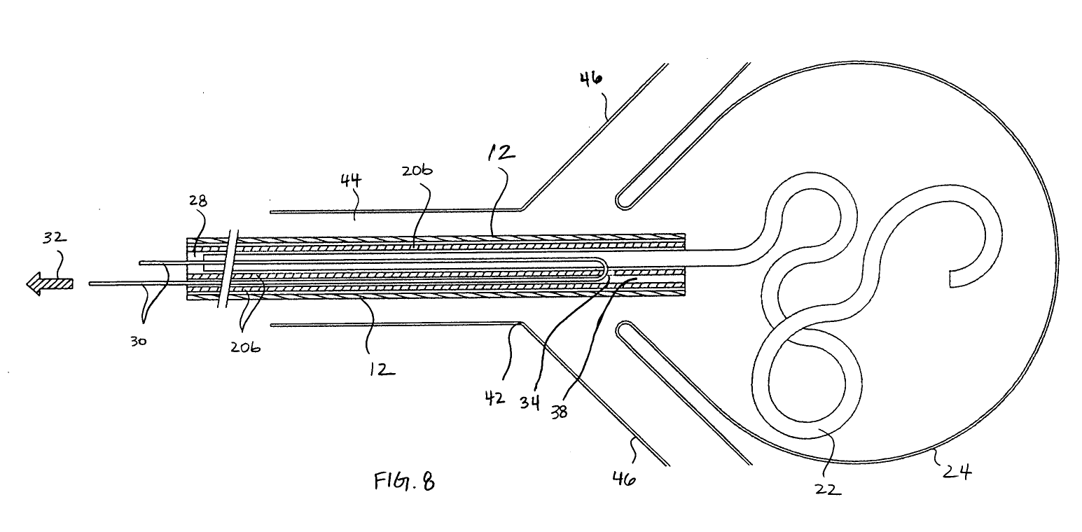

Referring now to FIG. 7, the double lumen delivery catheter (20c) of FIG. 5 is illustrated. However in this embodiment, a core member (40) is additionally embedded along a length of the occlusive element (22). The integration or inclusion of the core member (40) may be achieved during or after the occlusive element (22) extrusion process. The core member (40) may comprise metal materials, such as stainless steel, shape memory alloy, or superelastic metal, or polymer materials, such as nylon, polyurethane, polyimide, polyester, polypropylene, polyethylene, silk, PTFE (polytetrafluoroethylene), ePTFE (expanded polytetrafluoroethylene), PET (polyethyleneterephthalate), CRISTAMID®, GRILAMID®, PEBAX®. The core member (40) enhances the mechanical integrity, such as the tensile strength, of the occlusive element which in turn improves the desired advancement, positioning, and retractability of such occlusion materials at a treatment site. The core member (40) will generally have a length of at least about 0.5 cm, preferably in a range from about 2 cm to about 500 cm. Additionally, FIG. 7 illustrates that the reinforcing member 30 may be offset from a central axis of the occlusive element (22).

Referring now to FIG. 8, a method for intraluminal delivery of an occlusive element (22) into an aneurysm (24) at a bifurcated vessel juncture (42) using the delivery sheath (20b) of FIG. 4 is described. It will be appreciated that the present invention may be used in a variety of vessels, ducts, and cavities found in the body, and is not limited to bifurcated aneurysms. The delivery sheath (20b) may be slidably received within the catheter structure (12). The delivery sheath (20b) will generally have a length in a range from about 5 cm to about 300 cm, preferably from about 100 cm to about 250 cm. The delivery sheath (20b) is positioned in a body lumen 44 with a distal end thereof at the aneurysm (24) site at the bifurcated juncture (42). Once properly positioned, the reinforcing member (30) is pulled in a proximal direction as denoted by arrow (32) through the return lumen (38). In some instances, a pulling assembly may be coupled to a proximal end of the reinforcing member via the hub, such as a Y adaptor having insertion and return lumens, for automatic operation. Alternatively, pulling may be implemented simply by manual operation. In either embodiment, pulling in turn causes the occlusive element (22) to be advanced into the aneurysm (24).

The reinforcing member (30) of the present invention advantageously provides an advancing axial force along a length of the occlusive element (22) to prevent the occlusive element (22) from buckling and obstructing or clogging a distal end or tip of the delivery sheath (20b). Pulling (32) further comprises detaching the reinforcing member (30) from the occlusive element (22) at a distal end of the insertion lumen (34) so as to advance the occlusive element (22) distally of the delivery sheath (20b) for proper positioning at the aneurysm (24). The method further comprises hydrating the filament (22) prior to advancing the occlusive element (22) to the aneurysm (24), so as to form a polymeric gel. Additionally, in certain circumstances such as inadvertent advancement of the occlusive element (22) into non-target vessels (46), the occlusive element (22) may be retracted through the insertion lumen (28) from the vessel (46) and/or sheath (20b) via the reinforcing member (30).

Referring now to FIG. 9, an alternative system for intraluminal delivery of an occlusive element is illustrated. The system comprises a delivery catheter (12a), at least one occlusive element (22), and at least one reinforcing sheath (48). The delivery catheter (12a) generally comprises a dual lumen structure having both an insertion lumen (50) and return lumen (52) therethrough. The at least one occlusive element (22) is slidably received within the insertion lumen (50). The at least one reinforcing sheath (48) encompasses the occlusive element (22) along a length thereof and is slidably received within both the insertion (50) and return lumens (52). This design advantageously preserves the mechanical integrity of the occlusive element (22) without the need for auxiliary components, such as a delivery sheath.

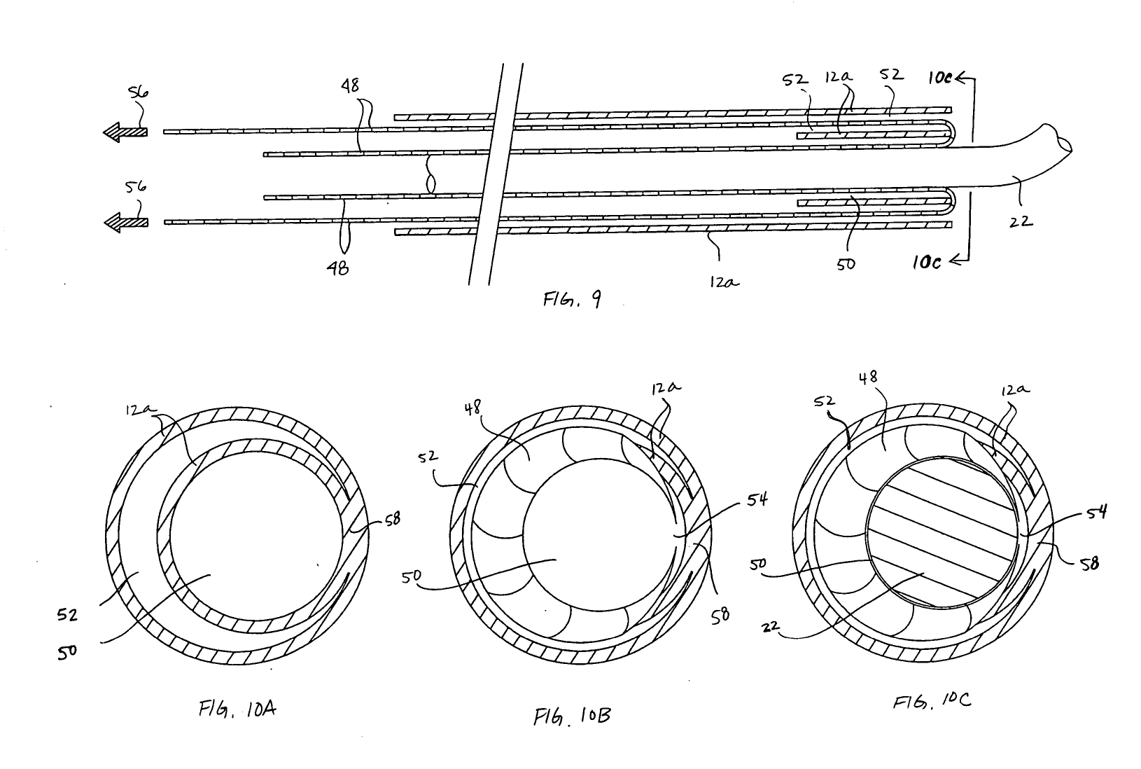

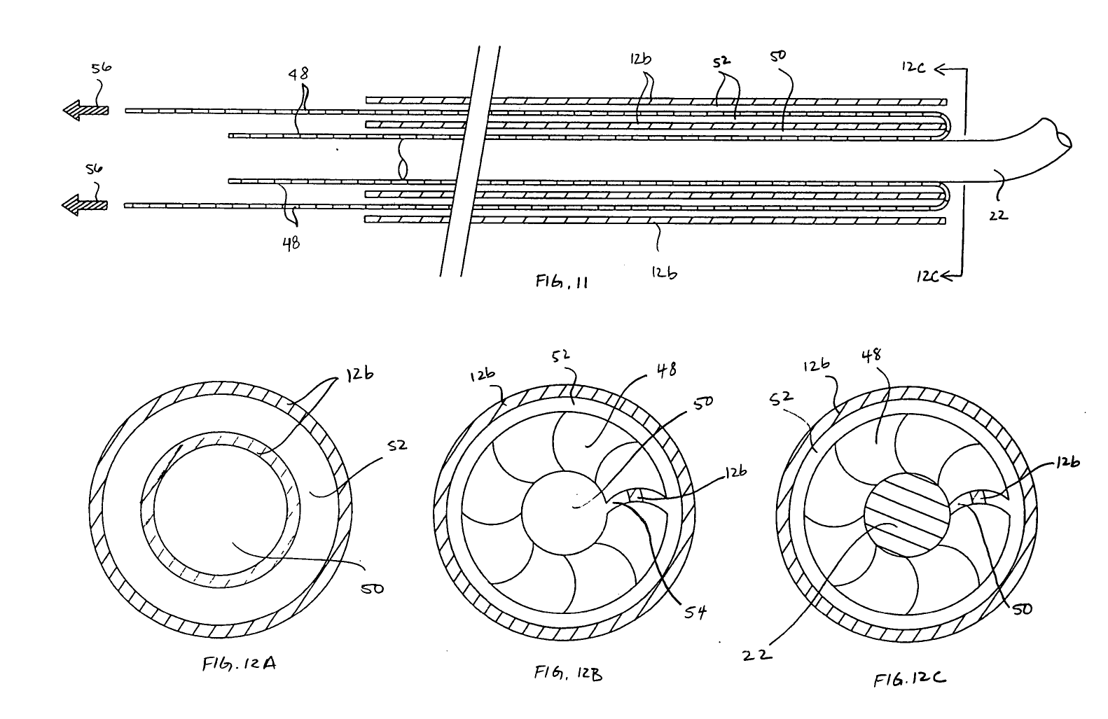

As shown in FIG. 10B and 10C, the reinforcing sheath (48) will have a longitudinal opening (54) along a length thereof so as to allow for detachment of the occlusive element (22) from the inverting sheath (48) as it is pulled in a proximal direction as denoted by arrows (56). The reinforcing sheath (48) may be formed from a variety of medical grade materials, including nylon, polyurethane, polyimide, polyester, polypropylene, polyethylene, silk, PTFE (polytetrafluoroethylene), ePTFE (expanded polytetrafluoroethylene), PET (polyethyleneterephthalate), CRISTAMID®, GRILAMID®, PEBAX®, and like threads or materials. The reinforcing sheath (48) may comprise a circumferential arc in a range from about 180 degrees to about 360 degrees, preferably from about 270 degrees to about 340 degrees. The reinforcing sheath (48) will have a length in a range from about 10 cm to about 600 cm, preferably from about 100 cm to about 500 cm. Generally, the length of the reinforcing sheath (48) is at least twice the length of the delivery catheter (12) so that the occlusive element (22) is properly advanced as the reinforcing sheath (48) is expulsed.

As shown in FIG. 10A, the insertion lumen (50) and return lumen (52) preferably form concentric tubular members that may have varying lengths. The delivery catheter (12a) will generally have a length in a range from about 5 cm to about 300 cm, preferably from about 50 cm to about 250 cm, and a diameter in a range from about 0.025 inch to about 0.250 inch, preferably from about 0.030 inch to about 0.100 inch. As illustrated in FIG. 9, the insertion lumen (50) and return lumen (52) may merge as interference between insertion and return of the reinforcing sheath (48) may be negligible. In such a case, walls of the insertion lumen (50) and return lumen (52) may form a bridge (58) at a distal end of the delivery catheter (12a) so that the lumens remain stationary with respect to each other.

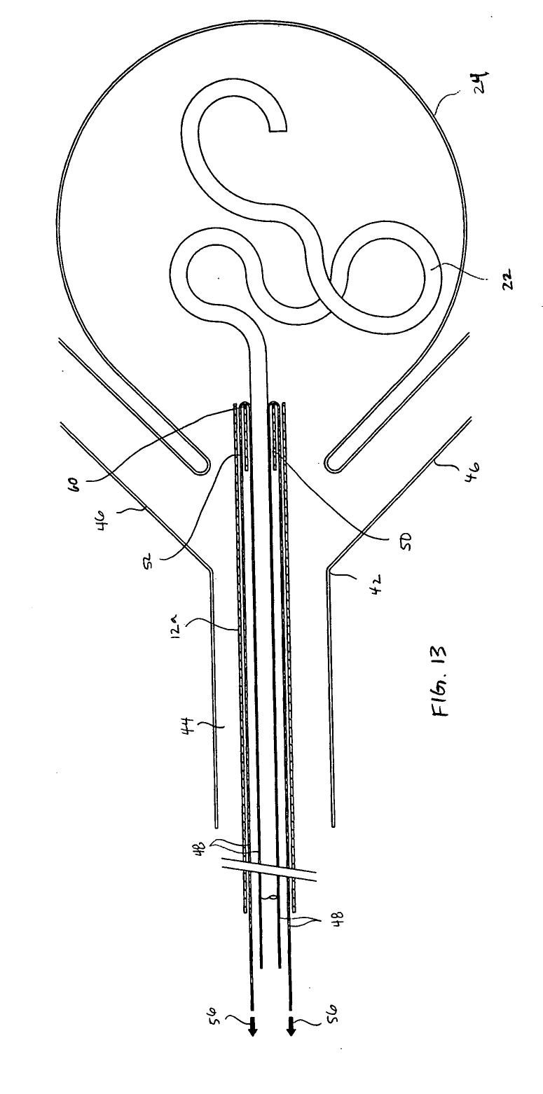

Referring now to FIGS. 11, 12A, 12B, and 12C, another double lumen delivery catheter (12b) is illustrated. The main distinction of the delivery catheter (12b) of FIG. 11 is that the insertion lumen (50) and return lumen (52) extend along a length of the catheter (12b). In such an embodiment, the insertion lumen and return lumen may form a bridge at a proximal end of the delivery catheter (not shown).

Referring now to FIG. 13, a method for intraluminal delivery of an occlusive element (22) into an aneurysm (24) at a bifurcated vessel juncture (42) using the delivery catheter (12a) of FIG. 9 is described. It will be appreciated that the present invention may be used in a variety of vessels, ducts, and cavities found in the body, and is not limited to bifurcated aneurysms. The delivery catheter (12a) is positioned in a body lumen (44) with a distal end (60) thereof at the aneurysm (24) site at the bifurcated juncture (42). Once properly positioned, the reinforcing sheath (48) is pulled in a proximal direction as denoted by arrows (56) through the return lumen (52). In some instances, a pulling assembly may be coupled to a proximal end of the reinforcing sheath via the hub, such as a Y adaptor having insertion and return lumens, for automatic operation. Alternatively, pulling may be implemented simply by manual operation. In either case, pulling in turn causes the occlusive element (22) to be advanced into the aneurysm (24).

As shown in FIG. 13, the reinforcing sheath (48) instead provides an advancing shear force to an outside surface of the occlusive element (22) along a length thereof to prevent the occlusive element (22) from buckling and obstructing or clogging a distal end or tip of the delivery catheter (12a). Pulling further comprises inverting the reinforcing sheath (48) at a distal end (60) of the insertion lumen (50) so as to deposit the occlusive element (22) distally of the delivery catheter (12a) for proper positioning at the aneurysm (24). Additionally, in certain circumstances such as inadvertent advancement into non-target vessels (46), the occlusive element (22) may be retracted. This may be accomplished by pulling proximally on the delivery sheath (48), noodle (22), and/or core member (40) through the insertion lumen (50). This causes the reinforcing sheath (48) to re-wrap onto the occlusive element (22) during retraction.

Although certain exemplary embodiments and methods have been described in some detail, for clarity of understanding and by way of example, it will be apparent from the foregoing disclosure to those skilled in the art that variations, modifications, changes, and adaptations of such embodiments and methods may be made without departing from the true spirit and scope of the invention. Therefore, the above description should not be taken as limiting the scope of the invention which is defined by the appended claims.

1. A method for intraluminal delivery of an occlusive element comprising:
positioning a delivery sheath in a body lumen, the sheath having at least an insertion lumen therethrough and containing at least one occlusive element and a reinforcing member coupled to the occlusive element along a length thereof;
pulling the reinforcing member in a proximal direction; and
advancing the occlusive element to a target site.

2. The method of claim 1, wherein the reinforcing member provides an advancing axial force along a length of the occlusive element.

3. The method of claim 1, wherein pulling comprises pulling the reinforcing member in a proximal direction over an outside surface of the delivery sheath.

4. The method of claim 1, wherein pulling comprises pulling the reinforcing member in a proximal direction through a return lumen of the delivery sheath.

5. The method of claim 1, wherein pulling further comprises detaching the reinforcing member from the occlusive element at a distal end of the insertion lumen so as to advance the occlusive element distally of the delivery sheath.

6. The method of claim 1, wherein the body lumen comprises a blood vessel.

7. The method of claim 1, wherein the target site comprises an aneurysm.

8. The method of claim 1, wherein the target site comprises vasculature of a tumor.

9. The method of claim 1, wherein the occlusive element comprises a polymeric filament.

10. The method of claim 9, further comprising hydrating the filament prior to advancing the occlusive element to the target site.

11. The method of claim 1, further comprising retracting the occlusive element through the insertion lumen.

12. A system for intraluminal delivery of an occlusive element comprising:
a delivery sheath having at least an insertion lumen and return lumen therethrough;
at least one occlusive element slidably received within the insertion lumen;
at least one reinforcing member coupled to the occlusive element along a length thereof and slidably received within both the insertion and return lumens.

13. The system of claim 12, wherein the insertion lumen and return lumen are concentric.

14. A system for intraluminal delivery of an occlusive element comprising:
a delivery sheath having at least an insertion lumen therethrough;
at least one occlusive element slidably received within the insertion lumen;
at least one reinforcing member coupled to the occlusive element along a length thereof and disposed over an outside surface of the delivery sheath.

15. The system of claim 12 or 14, wherein the reinforcing member provides an axial force along a length of the occlusive element.

16. The system of claim 12 or 14, wherein the reinforcing member is detachable from the occlusive element at a distal end of the insertion lumen so as to advance the occlusive element distally of the delivery sheath.

17. The system of claim 12 or 14, further comprising an opening in a distal wall of the insertion lumen.

18. The system of claim 12 or 14, wherein a length of the reinforcing member is at least twice the length of the delivery sheath.

19. The system of claim 12 or 14, wherein the reinforcing member comprises nylon, polyurethane, polyimide, polyester, polypropylene, polyethylene, silk, PTFE (polytetrafluoroethylene), ePTFE (expanded polytetrafluoroethylene), PET (polyethyleneterephthalate), CRISTAMID®, GRILAMID®, PEBAX® materials.

20. The system of claim 12 or 14, wherein the reinforcing member is centrally embedded within the occlusive element.

21. The system of claim 12 or 14, wherein the reinforcing member is offset from a central axis of the occlusive element.

22. The system of claim 12 or 14, wherein the occlusive element comprises a filament.

23. The system of claim 23, wherein the filament is hydratable.

24. The system of claim 23, wherein the filament comprises a polymeric gel.

25. The system of claim 12 or 14, further comprising a core member embedded along a length of the occlusive element.

26. The system of claim 25, wherein the core member comprises metal or polymer material.

27. The system of claim 12 or 14, further comprising a catheter which receives the delivery sheath.

28. The system of claim 27, wherein the delivery sheath and catheter form an integrated unit.

29. A method for intraluminal delivery of an occlusive element comprising:
positioning a delivery catheter in a body lumen, the catheter having at least an insertion lumen and return lumen therethrough and containing at least one occlusive element and a reinforcing sheath encompassing at least a portion of the occlusive element along a length thereof;
pulling the reinforcing sheath in a proximal direction through the return lumen; and
advancing the occlusive element to a target site.

30. The method of claim 29, wherein the reinforcing sheath provides an advancing shear force to an outside surface of the occlusive element along a length thereof.

31. The method of claim 29, wherein pulling further comprises inverting the reinforcing sheath at a distal end of the insertion lumen so as to advance the occlusive element distally of the delivery catheter.

32. The method of claim 29, wherein the body lumen comprises a blood vessel.

33. The method of claim 29, wherein the target site comprises an aneurysm.

34. The method of claim 29, wherein the target site comprises vasculature of a tumor.

35. The method of claim 29, wherein the occlusive element comprises a filament.

36. The method of claim 35, further comprising hydrating the filament prior to advancing the occlusive element to the target site.

37. The method of claim 35, wherein the filament comprises a polymeric gel.

38. The method of claim 29, further comprising retracting the occlusive element through the insertion lumen.

39. A system for intraluminal delivery of an occlusive element comprising:
a delivery catheter having at least an insertion lumen and return lumen therethrough;
at least one occlusive element slidably received within the insertion lumen;
at least one reinforcing sheath encompassing the occlusive element along a length thereof and slidably received within both the insertion and return lumens.

40. The system of claim 39, wherein the reinforcing sheath provides a shear force to an outside surface of the occlusive element along a length thereof.

41. The system of claim 39, wherein the reinforcing sheath is invertable at a distal end of the insertion lumen so as to advance the occlusive element distally of the delivery catheter.

42. The system of claim 39, wherein the reinforcing sheath has a longitudinal opening along a length thereof.

43. The system of claim 39, wherein the reinforcing sheath comprises a circumferential arc in a range from 270 degrees to 340 degrees.

44. The system of claim 39, wherein a length of the reinforcing sheath is at least twice a length of the delivery catheter.

45. The system of claim 39, wherein the reinforcing sheath comprises nylon, polyurethane, polyimide, polyester, polypropylene, polyethylene, silk, PTFE (polytetrafluoroethylene), ePTFE (expanded polytetrafluoroethylene), PET (polyethyleneterephthalate), CRISTAMID®, GRILAMID®, PEBAX® materials.

46. The system of claim 39, wherein the insertion lumen and return lumen are concentric.

47. The system of claim 46, wherein the insertion lumen and return lumen have different lengths.

48. The system of claim 47, wherein walls of the insertion lumen and return lumen are bridged to form an integrated unit.

49. The system of claim 39, wherein the occlusive element comprises a filament.

50. The system of claim 49, wherein the filament is hydratable.

51. The system of claim 49, wherein the filament comprises a polymeric gel.

52. The system of claim 39, further comprising a core member embedded along a length of the occlusive element.

53. The system of claim 52, wherein the core member comprises metal or polymer material.

54. A kit comprising:
an occlusive element delivery system; and
instructions to use the system for intraluminal delivery of the occlusive element according to claim 1 or 29.
